(19) United States
(12) Patent Application Publication
(10) Pub. No.: US 2009/0272377 A1
(43) Pub. Date: Nov. 5, 2009

(54) Inhalation-controlled nebulizer with oscillating baffle

(76) Inventor: Samuel David Piper, II, Carmichael, CA (US)

Correspondence Address: David Piper, P.O. Box 993, Carmichael, CA, 95609, US

(21) Appl. No.: 12/386,692

(22) Filed: Apr. 21, 2009

Related U.S. Application Data
(60) Provisional application No. 61/126,224, filed on May 1, 2008.

(51) Int. Cl. A61M 11/00

(52) U.S. Cl. 128/200.18

(57) ABSTRACT

The present invention is directed generally to a nebulizer for the formation of micro-droplets from liquid medicaments for respiratory patient treatment, and more specifically, to a baffled nebulizer wherein the baffle oscillates within the nebulizer. The oscillating baffle operates such that as a constant stream of liquid medicament strikes a target region within the oscillating baffle, at a first position the medicament stream forms a predominant fraction of macro-droplets, which are substantially recycled, and in a second position, the medicament stream forms a predominant fraction of micro-droplet respirable aerosol. The oscillating baffle moves between the first and second positions based upon the respiration of the patient, thus moving the oscillating baffle into the second position only when inhalation occurs, thereby preventing excessive waste of liquid medicament, and improving patient therapy.

### PARENT CASE TEXT

This application claims the benefit under 35 U.S.C. 119(e) of U.S. provisional applications Ser. No. 61/126,224 filed May 1, 2008, which is incorporated by reference herein in its entirety.

### BACKGROUND OF THE INVENTION

Nebulizers for producing aerosols of micro-droplets from liquid medicaments and presenting those aerosols for patient respiratory therapy are a well-known and practiced technology. A typical nebulizer design includes the basic elements of a gas inlet port, a respirable gas outlet, a liquid reservoir and a means for forming micro-droplets of the liquid within the reservoir. Early designs, such as represented by U.S. Pat. No. 3,097,645 and U.S. Pat. No. 3,762,409, both to Lester and incorporated by reference in their entireties herein, depict the basic elements of a typical constant-flow type nebulizer. Constant-flow type nebulizers create micro-droplets of liquid medicament based on an uninterrupted supply of pressurized gas coming through the gas inlet port and entraining liquid from the reservoir continually into an aerosol until such time the pressurized gas is stopped or the reservoir of liquid becomes empty. While representative simple nebulizers such as taught by Lester are capable of producing an aerosol, the efficiency of simply jetting an entrained liquid stream into a free space was not found to be adequate for creating micro-droplets of a consistent size and rate. U.S. Pat. No. 4,588,129 to Shanks, incorporated herein by reference in its entirety, addresses this consistent size and rate issue of the earlier Lester designs by further incorporating a fixed baffle having a convex target surface. In the Shanks nebulizer, a liquid entrained jet stream strikes upon the convex target surface of the baffle and the impact thereof allows for the momentum imbued within the liquid stream to mechanically act upon the stream and cause the creation of smaller, more readily inhaled micro-droplets at a higher rate.

The Lester and Shanks nebulizers greatly advanced the art or aerosol formation, however, due to their continuous aerosol formation mode of operation, much of the liquid medicament was formed into an aerosol, which is then lost from the device during patient exhalation and idle operation of the device. Loss of aerosolized medicament to the environment is deleterious as there is a decrease in therapeutic value to the patient resulting from reduced dosing, as well as, contamination of the immediate atmospheric environment and inadvertent dosing of individuals not requiring treatment. Improvements were then made to alter nebulizer performance such that the creation of micro-droplets through aerosolization occurred only when the patient being treated was inhaling through the nebulizer. Published U.S. Patent Application 2003/0136399 to Foley, et al., teaches a means for a nebulizer, which creates a constant micro-droplet aerosol within a closed chamber, which is released through operation of a valve. Published U.S. Patent Application 2002/0157663 to Blacker, et al., seeks to control aerosol production through patient inhalation completing the path from the liquid reservoir to the entrainment orifice and thereby allow liquid to entrain into the pressurized gas. U.S. Pat. No. 7,080,643 to Grychowski, et al., utilizes a gas diverter, which moves into and out of position wherein pressurized gas is directed across liquid transfer conduits and the vacuum created thereby causes liquid to be drawn through the transfer conduits and entrained into the gas flow.

While current inhalation controlled nebulizers are effective, utilizing multiple control components and complex routing, the spirit of a simple and easy to operate device as was captured in the earlier continuous jet nebulizers has been lost. Therefore, a need exists for an inhalation-controlled device that is capable of producing micro-droplets at a rapid and constant rate, wherein the device is greatly simplified, thus improving reliability, ease of manufacture and ready use by individuals.

### BRIEF SUMMARY OF THE INVENTION

The present invention is directed generally to a nebulizer for the formation of micro-droplets from liquid medicaments for respiratory patient treatment, and more specifically, to a baffled nebulizer wherein the baffle oscillates within the nebulizer such that as a constant stream of liquid medicament strikes a target region within the oscillating baffle, at a first position in the oscillation the medicament stream forms a macro-droplet and in a second position in the oscillation the medicament stream forms a micro-droplet. An entrainment orifice within the nebulizer utilizes pressurized gas to draw in liquid medicament from a reservoir and to entrain that liquid medicament into a continuous high velocity stream. The high velocity liquid entrained jet is oriented such that an optimization point is achieved at a defined distance in front of the entrainment orifice. When the oscillating baffle is in a first position, the target region within the baffle is at a point that is outside the optimization point of the liquid jet stream. As the liquid jet stream passes the optimization point of the jet, the diffusing jet then strikes the target region of the oscillating baffle. When the liquid jet stream impacts upon the target region, and through the combined actions of jet dispersion and loss of jet momentum, the liquid forms macro-droplets; macro droplets which then return to the reservoir for re-entrainment. When the oscillating baffle is in a second position, the target region within the baffle is at a point that is within the optimization point of the liquid jet stream. As the liquid jet stream comes to the optimization point of the jet, the jet strikes the target region of the oscillating baffle. When the liquid jet stream impacts upon the target region at the optimization point, and through the combined actions of minimal jet dispersion and high jet momentum, the liquid forms micro-droplets; micro droplets which are then drawn into the pulmonary space of a patient. The oscillating baffle moves between the first and second positions based upon the respiration of the patient, thus moving the oscillating baffle into the second position only when inhalation occurs, thereby preventing excessive waste of liquid medicament, and improving patient therapy.

### DESCRIPTION OF THE SEVERAL VIEWS OF THE DRAWING(S)

The invention will be more easily understood by a detailed explanation of the invention including drawings. Accordingly, drawings, which are particularly suited for explaining the inventions, are attached herewith; however, it should be understood that such drawings are for descriptive purposes only and as thus are not necessarily to scale beyond the measurements provided. The drawings are briefly described as follows:

FIG. 1 is graphic representation of the present invention, wherein the baffle distance (“BD”) of the oscillating baffle target surface relative to the entrainment orifice is depicted.

FIG. 2 is an exploded perspective view of an inhalation-controlled nebulizer with oscillating baffle.

FIG. 3 is across sectional side view of an inhalation controlled nebulizer with oscillating baffle.

FIG. 4 is a cross sectional side view of an inhalation-controlled nebulizer wherein the oscillating baffle is in a first, macro-droplet forming position.

FIG. 5 is a cross sectional side view of an inhalation-controlled nebulizer wherein the oscillating baffle is in a second, micro-droplet forming position.

FIG. 6 is a side view of an oscillating baffle.

FIG. 7 is a side view of an oscillating baffle with an optional circumferential baffle flange.

FIG. 8 is a graph showing liquid aerosolization performance of a representative embodiment in terms of baffle distance BD versus optimized baffle distance BD(sub)OPT.

### ASSIGNMENT OF COMPONENT NUMBERING

Nebulizer unit 4, upper chamber 10, lower chamber 12, liquid reservoir 14, air chimney 16, gas inlet port 20, inhalation port 22, liquid ejection nozzle 24, entrainment orifice 26, liquid transfer channels 28, jet orifice 30, oscillating baffle 40, target surface 42, air inlet portal 44, retention ring 46, liquid backflow guard 48, baffle flange 50, aerosolization limiter 52, travel limiter 54, and override plunger 60.

### DETAILED DESCRIPTION OF THE INVENTION

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated.

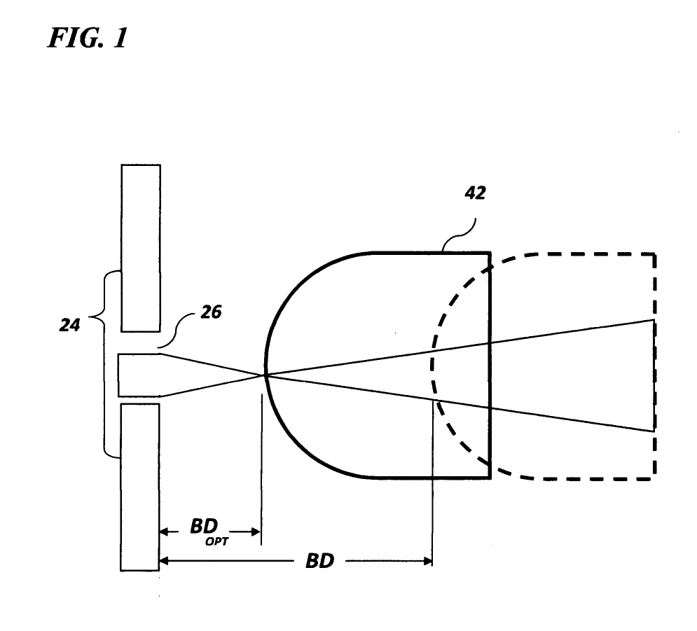

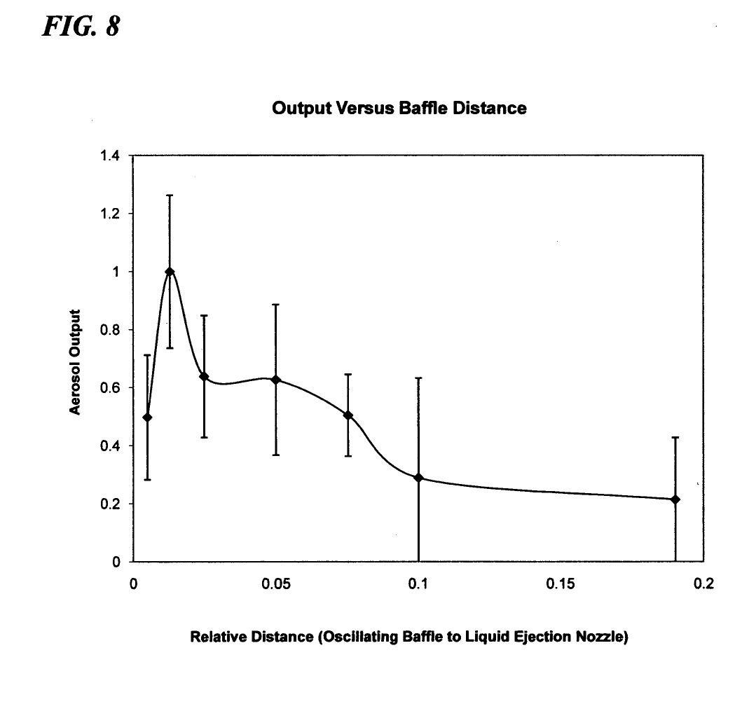

Referring more specifically to the figures, for illustrative purposes the present invention is embodied in the apparatus generally shown in FIG. 1 through FIG. 7 and device performance graph provided in FIG. 8.

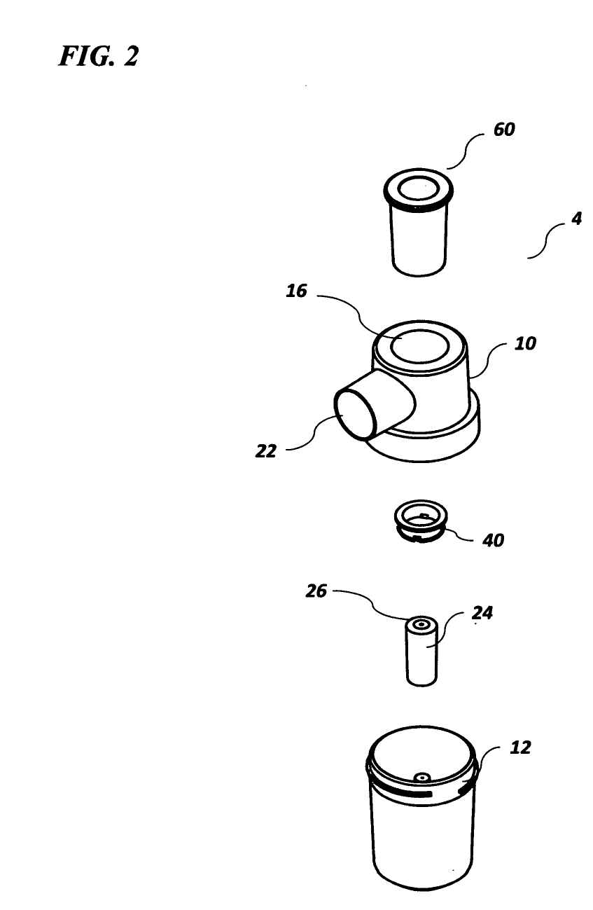

A graphical representation of the functional attribute embodied in the present invention is depicted in FIG. 2 to FIG. 5 and will be further disclosed in detail in the following description. An entrainment orifice 26 within a nebulizer 4 utilizes pressurized gas to draw in liquid medicament from a liquid reservoir 14 and to entrain that liquid medicament into a continuous high velocity stream. The high velocity liquid entrained jet is oriented such that an optimized focal point is achieved at a defined distance (“BD(sub)OPT)” from the entrainment orifice (shown in detail in FIG. 1). When a provided oscillating baffle 40 is in a first position, a target surface 42 within the oscillating baffle 40 is at a baffle distance (“BD”) that is outside the optimization point BD(sub)OPT of the liquid jet stream (where BD is greater than or less than about 10% of BD(sub)OPT, and preferably greater than or less than about 5% of BD(sub)OPT). As the liquid jet stream is outside the optimization point BD(sub)OPT of the jet as it strikes the target surface 42 of oscillating baffle 40, the liquid jet stream, through the combined actions of jet dispersion and loss of jet momentum, preferentially forms an increased fraction of macro-droplets, macro-droplets which then return to liquid reservoir 14 for re-entrainment. When oscillating baffle 40 is in a second position, a target surface 42 within the oscillating baffle 40 is at a point that is within the optimization point BD(sub)OPT of the liquid jet stream. As the liquid jet stream comes to the optimization point of the jet, the jet strikes the target region 42 of oscillating baffle 40. The liquid jet stream impacting upon target surface 42, and through the combined actions of minimal jet dispersion and high jet momentum forming micro-droplets of liquid medicament of which an increased fraction of said micro-droplets are then drawn out of nebulizer 4 and into the pulmonary space of a patient (not shown). The oscillating baffle 40 moves cyclically between the first and second positions based upon the respiration of the patient, thus moving the oscillating baffle 40 into the second position only when inhalation occurs, thereby preventing excessive waste of liquid medicament, and improving patient therapy.

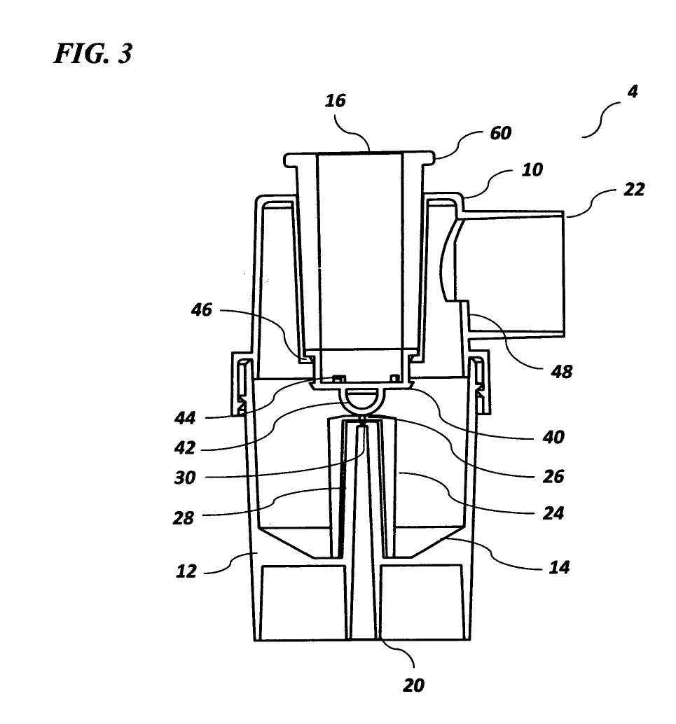

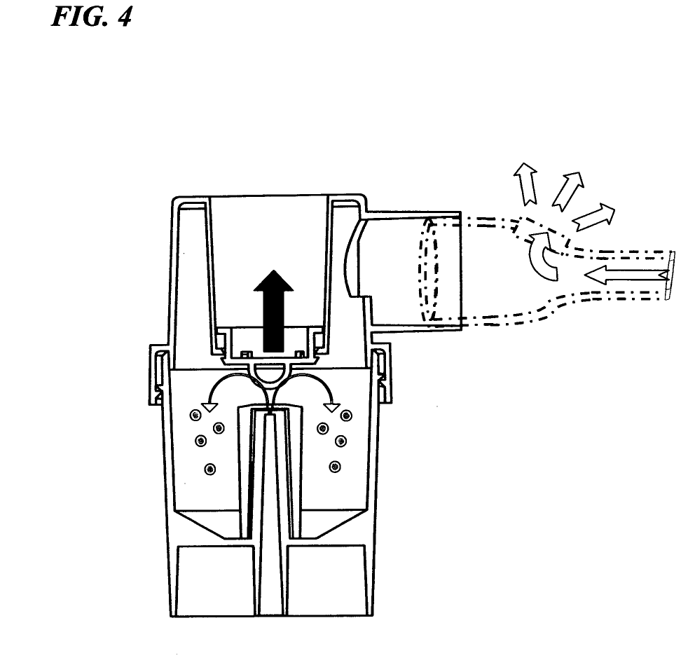

Returning to FIG. 2, therein is depicted nebulizer unit 4. Nebulizer unit 4 is comprised of an upper chamber 10 and a lower chamber 12. Upper chamber 10 has therein an air chimney 16 for allowing ambient air to be drawn through chamber 10 into oscillating baffle 40 and an inhalation port 22 having a liquid backflow guard 48, which is in fluid communication with a patient through a suitable mouth piece (depicted in FIGS. 3 and 4) or endotracheal tube connection (not shown). Suitable mouthpieces include, but are not limited to, such commercially available devices complying with a standard 22 millimeter ISO ID “Mouth Piece Connection”, and preferably include mouth pieces having an exhalation valve and an exit angle from inhalation port 22 of 0 degrees (i.e. straight continuation of inhalation port 22) to 45 degrees (as measured from an axis taken from the center and extending parallel in the direction of inhalation port 22). Lower chamber 12 has therein a liquid reservoir 14 and a gas inlet port 20 (more readily visible in FIGS. 3, 4, and 5). Gas inlet port 20 extends from an area exterior to lower chamber 12 whereby it is attached to a pressurized gas supply (not shown) and passes through liquid reservoir 14, into liquid ejection nozzle 24 and concluding at an area proximal to upper chamber 10. In the embodiment shown, upper chamber 10 and lower chamber 12 are releasably affixed to one another so that liquid medicament can be introduced into liquid reservoir 14. It is within the purview of the present invention that a liquid addition portal can be provided for introduction of liquid medicament, and in such case, upper chamber and lower chamber may be permanently affixed at the time of manufacture.

Liquid ejection nozzle 24 comprises entrainment orifice 26, liquid transfer conduit 28, and jet orifice 30. In accordance with the liquid ejection nozzles taught by Lester in the aforementioned and incorporated patents of reference, as pressurized gas issues from entrainment orifice 30 into entrainment orifice 26, liquid from reservoir 14 is drawn up liquid transfer conduit 28. As liquid is drawn through liquid transfer conduit 28, it passes through a flow control point (typically about 0.010 inch in height) and then into direct contact with, and becomes entrained within, the gas issuing from jet orifice 30 and is forcibly ejected from entrainment orifice 26 as a focused continuous stream of liquid entrained gas. Entrainment orifice 26 has a geometry sufficient to focus the liquid entrained jet at an optimization point BD(sub)OPT within the confines defined by nebulizer 4 and within the travel of oscillating baffle 40. It is within the purview of the present invention that one or more liquid entrained gas jet may be formed by liquid ejection nozzle 24.

Within a central region of upper chamber 10, there extends downwardly air chimney 16. Air chimney 16 may be either an element integral to upper chamber 10 or separate element affixed to a central void within upper chamber 10. In a preferred embodiment, air chimney 16 is generally round in cross section taken at a point parallel to a point of junction with lower chamber 12. The air chimney 16 extends into a central void of upper chamber 10 and has a distal point that is proximal to liquid ejection nozzle 24. At the distal point of air chimney 16 therein is a retention ring 46 that acts upon oscillating baffle 40 to prevent oscillations from exceeding the bound of oscillating travel.

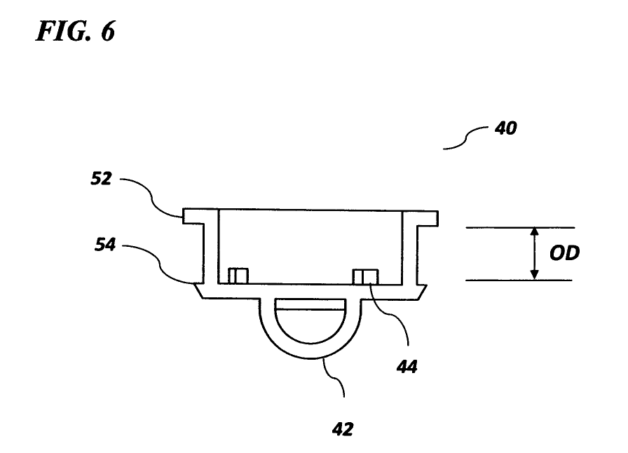

Oscillating baffle 40 is comprised of target surface 42 and air inlet portal 44. Within the design of oscillating baffle 40 there is an aerosolization limiter 52 and a travel limiter 54 (detail in FIG. 6). Aerosolization limiter 52 is separated from travel limiter 54 by an oscillation distance (“OD”). During operation of the nebulizer unit 4, the oscillating baffle 40 will move into a first position wherein travel limiter 54 will come within proximity, and may even directly engage, retention ring 46. When the patient inhales, thereby reducing the pressure within nebulizer unit 4, oscillating baffle 40 will move in the direction of liquid ejection nozzle 24 and will come to a second position whereby aerosolization limiter 52 will directly engage on retention ring 46. Air inlet portal 44 can either of a predetermined finite shape and area, as well as being adjustable through use of such devices as an interdigitated disk having corresponding portals which coincide with the profile of the air inlet portal geometry and which may be rotated so that the resulting port shape and/or area can be increased or decreased. To further control the movement of oscillating baffle 40, a biasing member (not shown) such as a spiral wrapped spring can be used to bias the oscillating baffle 40 in either the direction of the aerosolization limiter 52 or the travel limiter 54.

When the travel limiter 54 of oscillating baffle 40 is proximal to or engaged with retention ring 46, air inlet portal 44 is partially or fully occluded to introduction of ambient air by the physical presence of retention ring 46 itself and/or air chimney 16. Air inlet portal 44 is exposed to a greater extent as oscillating baffle 40 moves to the second position by reduction in pressure within nebulizer 4 by the patient inhaling, thus allowing introduction of air into the inner aspect of the void defined within upper chamber 10 and lower chamber 12. The ambient air coming through air portal 44 provides the necessary flow, in conjunction with the air pressure being provided by gas inlet 20, to provide the patient sufficient inspiratory volume and rate. Further, without being constrained to specific theory, it is believed that ambient air drawn through air inlet portal 44 allows for rapid momentary evaporation of micro-droplets created by impact on target surface 40 during patient inhalation, thus improving the respirable volume of micro-droplets formed.

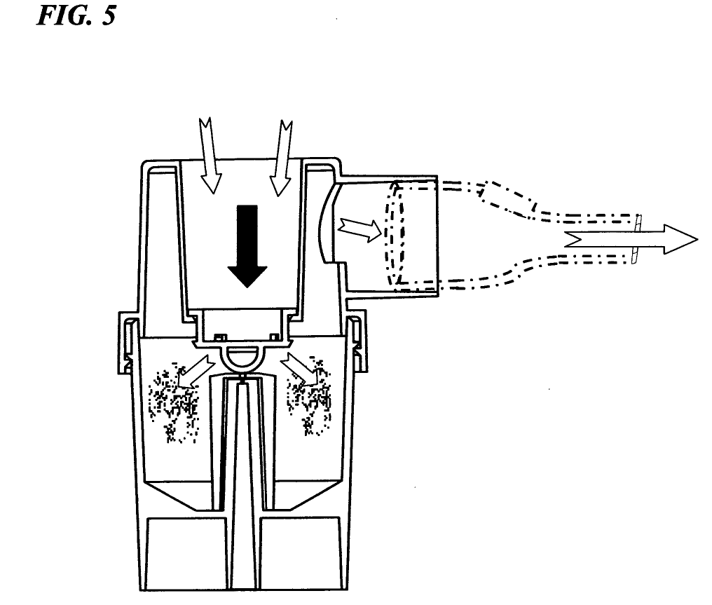

Upon oscillating baffle 40 there is a target surface 42. Target surface 42 may have a simple geometric or radiused cross sectional profile as well as compound combinations of differing geometric and/or radiused cross sectional profiles. In a preferred embodiment, target surface is of a convex or hemispherical cross sectional profile. When oscillating baffle 40 is in the second or aerosolization position (FIG. 5), the target surface is located at a finite distance from liquid ejection nozzle 24. The finite distance is specifically controlled such that target surface 42 is at the optimization point of liquid ejection nozzle 24 when the oscillating baffle 40 is in this position. The liquid jet stream then impacts upon target surface 42, and through the combined actions of minimal jet dispersion and high jet momentum, the liquid forms into an increased fraction of micro-droplets; micro droplets capable of being drawn out of upper chamber 10 through inhalation port 22 and into the pulmonary space of the inhaling patient. At such time as patient inhalation ceases, oscillating baffle returns to the first position constrained maximally by travel limiter 54 (FIG. 4). The distance oscillating baffle 40 moves will be at or about the oscillation distance, OD. As oscillating baffle 40 moves the oscillation distance, target surface 42 is moved away from the optimization point created by continuous liquid entrained jet ejecting from liquid ejection nozzle 24. The liquid jet stream continues to impact upon target surface 42, and through the combined actions of jet dispersion and loss of jet momentum, the liquid forms macro-droplets; macro droplets which then condense and return to liquid reservoir 14 in lower chamber 12 for repeat conduction through liquid transfer conduit 28 and re-entrainment in the gas stream issuing from jet orifice 30.

If the inhalation triggered performance of nebulizer unit 4 is not desired, it is possible to override manually the oscillating baffle 40 through insertion of a hollow cylindrical plunger 60. Override plunger 60 causes oscillating baffle 40 to move to the point aerosolization limiter 52 is fully engaged on retention ring 46.

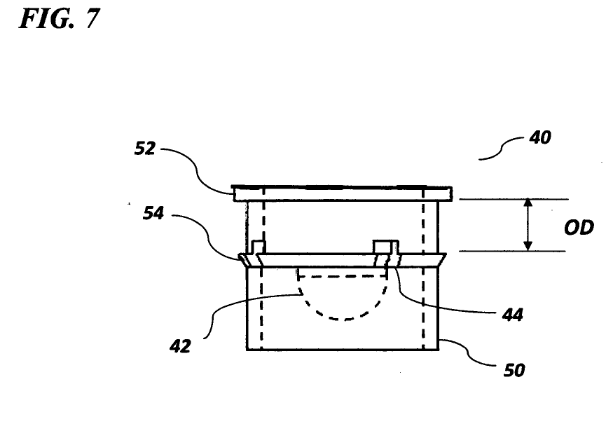

As shown in FIG. 7, therein is depicted an oscillating baffle 40 having the further inclusion of a baffle flange 50 around target surface 42. Baffle flange 50 can be advantageous in creating a smaller contact or wetting surface than that presented by the inner surfaces of upper chamber 10 and lower chamber 12, and thus reduces wet-out losses of liquid medicament. Baffle flange 50 may circumscribe target surface 42 in part or whole and may include straight, curved, or combined profiles as it extends downwardly from oscillating baffle 40. It is also within the purview of the present invention that air inlet portals 44 may be used to vent into the area circumscribed by baffle flange 50 to create additional turbulent flow and mixing of aerosolized micro-droplets into the gaseous flow being inhaled by the patient.

In practice with a preferred embodiment, nebulizer unit 4 is supplied gas to inlet port 20 at a pressure of 35 psig at a flow rate of between 1 and 15 liters of gas per minute, with the range of 5 to 12 liters per minute inclusively being preferred and the range of 8 to 11 liters per minute inclusively being most preferred. The gas issues through a jet orifice having a diameter in the range of 0.024 inch (plus or minus 10%). One or more liquid transfer conduits 28 are provided in liquid ejection nozzle 24 so that a volume of liquid medicament in the range of between 10 and 50 micro liters inclusively can be provided for aerosolization. The liquid entrain gas stream ejects from entrainment orifice 26 wherein entrainment orifice 26 has an orifice having a diameter in the range of 0.078 (plus or minus 10%). When operating in the above format, it is possible to complete a dosage of liquid medicament in less than three minutes at a supply pressure of 35 psig and a nominal flow rate of 10 liters per minutes.

The general construction of functional elements of nebulizer unit 4, includes thermoset and thermoplastic polymers as well as alloys and blends within those plastic families. Additional performance and aesthetic modifying chemistries can be incorporated during manufacture or after component or device fabrication. Of particular interest, polymers having specific surface energies can be used in different aspects of nebulizer unit 4 depending upon the degree of liquid medicament wet-out is desired. The nebulizer unit 4 of the present invention is not constrained by the mode of manufacture and may include known or developed methods in moulding and machining technology.

### EXAMPLE

A first embodiment nebulizer device in accordance with the present invention was fabricated and tested.

Device Dimensions:
Unit height: 3.75 inches
Unit width/diameter: 1.88 inches
Osciflation Distance: 0.17 inches
Inhalation port dimensions: 22 mm ISO ID “Respiratory Conduction Mouthpiece”
Liquid reservoir volume ranges: 3.0 milliliters
Air Chimney Inside Diameter: 0.90 inches
Override Inside Diameter: 0.80 inches

Device Performance:
Range of flow rates: 2 to 28 liters/minute
Normalized Aerosolized Liquid Rate v. Baffle Distance from Nozzle Reference FIG. 8

“Macro-droplets” are defined herein as being an individual unit of liquid medicament having an average diameter of greater than 10.0 micrometers and being in a predominant fraction of greater than 50% of the aerosolized liquid volume relative to the chamber volume of the nebulizer. “Micro-droplets” are defined herein as being an individual unit of liquid medicament having an average diameter of less than or equal to 10.0 micrometers and being in a predominant fraction of greater than 50% of the aerosolized liquid volume relative to the chamber volume of the nebulizer.

For the purposes of general background in aerosol technology and as indicia for the level of understanding resident in one skilled in the art of aerosol technology, the following references are incorporated by reference in their entireties as nonessential matter: “Aerosol Technology”, Hinds, 1982 by Wiley and Sons, ISBN 0-471-087726-2; “Inhalation Aerosols”, First Ed. Hickey, 1996 by Informa Healthcare, ISBN 0-8247-9702-7; and “The Mechanics of Aerosols”, Fuchs et al., 1989 by Dover Publications, ISBN 0-486-66055-9.

Although the description above contains many details, these should not be construed as limiting the scope of the invention but as merely providing illustrations of some of the presently preferred embodiments of this invention. Therefore, it will be appreciated that the scope of the present invention fully encompasses other embodiments, which may become obvious to those skilled in the art. In the appended claims, reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the disclosure and present claims. Moreover, it is not necessary for a device or method to address every problem sought to be solved by the present invention, for it to be encompassed by the disclosure and present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A nebulizer comprising of:
a. an upper chamber having therein an air chimney and an inhalation port for conduction of a gaseous suspension of aerosolized medicament to a patient;
b. a lower chamber having therein a gas inlet port adapted to receive pressurized gas and the ability to retain a liquid medicament within a liquid reservoir;
c. a liquid ejection nozzle in fluid communication with both of said gas inlet port and said liquid reservoir and which produces a liquid entrain gas jet when pressurized gas and liquid medicament are provided;
d. an oscillating baffle within said air chimney which is capable of oscillating from a first position distal to said nozzle to a second position proximal to said nozzle;
wherein said oscillating baffle in said first position allows for liquid entrained gas from said liquid ejection nozzle to impact upon said oscillating baffle and for an increased fraction of the liquid medicament to return to said liquid reservoir in said lower chamber;
wherein said oscillating baffle in said second position allows for liquid entrained gas from said nozzle to impact upon said oscillating baffle and to thereby create a gaseous suspension of an increased fraction of aerosolized liquid medicament in said upper chamber;
whereupon respiration by said patient causes said oscillating baffle to oscillate from said first position during a non-inhalation event to said second position during an inhalation event.

2. A nebulizer as in claim 1, wherein said oscillating baffle further comprises a target surface.

3. A nebulizer as in claim 2, wherein said target profile is convex profile.

4. A nebulizer as in claim 1, wherein said oscillating baffle further comprises a means for limiting an oscillation distance thereof.

5. A nebulizer as in claim 1, wherein said oscillating baffle further comprises an air inlet portal.

6. A nebulizer as in claim 1, wherein said liquid ejection nozzle focuses said liquid entrain gas jet to impact said oscillating baffle at a second distance that is an optimized focal point to produce micro-droplets.

7. A nebulizer comprising of:
a. an upper chamber having therein a vertically oriented air chimney and an inhalation port for conduction of a gaseous suspension of aerosolized medicament to a respiring patient;
b. a lower chamber having therein: a gas input port located centrally therein adapted to receive pressurized gas, extending from the base in an upward orientation, and a liquid reservoir;
c. a liquid ejection nozzle having a spray orifice in fluid communication with said gas inlet port and with said liquid reservoir, wherein said liquid reservoir communicates liquid medicament from the reservoir to the spray orifice by way of a liquid transfer conduit;
d. an oscillating baffle within said air chimney which is capable of oscillating in a linear path through said air chimney from a first distance distal to said liquid ejection nozzle to a second distance proximal to said liquid ejection nozzle;
wherein application of pressurized gas to said gas input port causes liquid medicament to move from the liquid reservoir and to mix into said pressurized gas to form a liquid entrained air jet issuing from said liquid ejection nozzle;
wherein said oscillating baffle in said first distance is impacted upon by said liquid entrained air jet forming macro-droplets of liquid medicament of which an increased fraction of said macro-droplets then return to said liquid reservoir in said lower chamber;
wherein said oscillating baffle in said second distance is impacted upon by said liquid entrained air jet forming micro-droplets of liquid medicament of which an increased fraction of said micro-droplets are conveyed to said patient through said outlet port;
whereupon respiration by said patient causes said oscillating baffle to oscillate from said first position during a non-inhalation event to said second position during an inhalation event.

8. A nebulizer as in claim 7, wherein said oscillating baffle further comprises a target surface.

9. A nebulizer as in claim 8, wherein said target surface is convex.

10. A nebulizer as in claim 7, wherein said oscillating baffle further comprises a baffle flange.

11. A nebulizer as in claim 7, wherein said macro droplets are greater than about 10 micrometers in diameter.

12. A nebulizer as in claim 7, wherein said micro droplets are equal to or less than about 10 micrometers in diameter.

13. A nebulizer as in claim 7, wherein said liquid ejection nozzle has a spray orifice of between 0.010 and 0.10 inch.

14. A nebulizer as in claim 7, wherein said first distance is within the range of 0.01 to 0.10 inch from said oscillating baffle to said entrainment jet.

15. A nebulizer as in claim 7, wherein said second distance is less than 90% of said first distance.

16. A nebulizer as in claim 7, wherein said second distance is greater than 110% of said first distance.

17. A method of forming medicament micro-droplet entrained gas for patient respiratory therapy comprising of:
a. Ejecting a pressurized jet of gas having entrained therein a liquid medicament;
b. Impinging said pressurized jet of liquid entrained gas against a oscillating baffle;
c. Moving said oscillating baffle between one of at least two distances in response to the respiratory action of a patient;
wherein at a first distance, said impingement of said pressurized jet of liquid entrained gas jet creates macro-droplets of liquid medicament of which an increased fraction of said macro-droplets then return for re-entrainment into said pressurized jet of liquid entrained gas; and
wherein at a second distance, said impingement of said pressurized jet of liquid entrained gas creates micro-droplets of liquid medicament of which an increased fraction of said micro-droplets are conveyed to said patient.

18. A method of forming medicament micro-droplet entrained gas as in claim 17, wherein movement of said oscillating baffle to said second distance is induced by inhalation of said patient.

19. A method of forming medicament micro-droplet entrained gas as in claim 17, wherein movement of said oscillating baffle is overridden by insertion of a plunger.

20. A method of forming medicament micro-droplet entrained gas as in claim 17, wherein a reduced quantity of medicament is dosed to the immediate atmospheric environment when said oscillating baffle is at said first distance.
